(19) United States
(12) Patent Application Publication
(10) Pub. No.: US 2004/0006387 A1
(43) Pub. Date: Jan. 8, 2004

(54) Intraocular lens

(76) Inventor: Charles David Kelman, Boca Raton, FL (US)

Correspondence Address: DARBY & DARBY P.C., 805 Third Avenue, New York, NY, 10022, US

(21) Appl. No.: 10/188,414

(22) Filed: Jul. 3, 2002

(51) Int. Cl. A61F 2/16

(52) U.S. Cl. 623/6.36; 623/6.43; 623/6.44

(57) ABSTRACT

An intraocular lens is provided and includes a deformable optic; a pair of support posts spaced circumferentially about the optic and extending outwardly therefrom; and a pair of retaining elements for securing the lens portion within an anterior chamber of the eye by grasping a portion of an anterior surface of iris tissue. Each retaining element has flexible first and second pincer tips that are normally in an abutting or a close abutting relationship when the retaining element is in a closed position. Each retaining element is secured to the anterior surface by opening the first and second pincer tips and pinching the anterior surface of the iris tissue. The optic is coupled to the retaining elements by disposing the support posts within openings or slots that are defined in the retaining elements. The coupling between the posts and the retaining elements is such that the optic is not only securely held within the anterior chamber but also is easily removable and adjustable.

### TECHNICAL FIELD

The present invention relates generally to intraocular lenses and more particularly, to a deformable intraocular lens and a method of anchoring the deformable intraocular lens.

### BACKGROUND

In the human eye, the natural lens is positioned behind the pupil and iris, and functions to focus light entering the eye onto the retina located at the rear of the eye. A healthy lens is a biconvex, highly transparent structure made of slender, curved rod-shaped ectodermal cells in concentric lamellae surrounded by a thin capsule. The lens capsule is supported at its periphery by suspensory ligaments, called zonules that are continuous with the ciliary muscles. Through controlled contraction of the ciliary muscles, the shape of the lens capsule may be altered, thereby changing the effective focal length of the lens and aiding in sharply focusing the incoming image light onto the retina.

Due to any number of conditions, vision can deteriorate over time and more specifically, the normal refraction of eye can deteriorate and necessitate that some type of corrective measure be taken by the patient if a correction in vision is desired. There are a number of different types of corrective measures that can be taken with some being less invasive than others. For example and assuming that the desired corrective measure is appropriate for the patient and will result in a correction of the patient's vision, the patient can simply choose to wear eye glasses. While eye glasses improve the patient's vision, there are a number of associated disadvantages, namely that eye glasses provide a limited field and disturbed peripheral vision. As an alternative, the patient can elect to wear contact lenses, which generally provide better peripheral vision but cannot always be tolerated by the eye. Other surgical corrective measures are available, such as correction with radial keratotomy, excimer laser, corneal inlays, epikeratophakia or keratomileusis. However, these corrective measures also each have associated disadvantages, ranging from poor optical correction, complicated and costly procedures, and long term uncertainty as to the results.

The use of intraocular lenses for refractory surgery (myopia, hypermetropia, presbyopia and astigmatism correction) in the phakic eye is well known. For example, U.S. Pat. No. 5,192,319 to Worst discloses an intraocular refractive lens having an optical portion with two pairs of pincer arms extending outwardly from the optical portion with one pair of pincer arms being formed at one end of the optical portion and the other pair of pincer arms being formed at the other end of the optical portion. The pincer arms grasp iris tissue to effectively secure the intraocular lens to the eye and locate the optical portion in a desired portion. While this intraocular lens provides some advantages, it also has a number of associated disadvantages. First, an incision needs to be formed in the eye in order for the intraocular lens to be received within the eye. Because of the makeup of the lens in the '319 patent, the dimensions of the incision are greater than those desired and it is therefore preferred to reduce the dimensions of the incision in order for the procedure to be as least invasive as possible.

Second, the manner of anchoring one end of the intraocular lens to the iris tissue and then anchoring the other end to the iris tissue, while maintaining the optical lens portion in its correct position, can be cumbersome since the intraocular lens is a unitary structure. More specifically, the intraocular lens of the '319 patent is inserted through an incision formed in the eye and one pair of the pincer arms are opened to create a gap therebetween. The pincer arms grasp the iris tissue with the tissue being received between the pincer arms within the gap. After the first pincer arms grasp the iris tissue, the surgeon is required to carefully manipulate the whole intraocular lens through the incision to locate the optical portion in the desired location within the eye above the natural human lens and to also securely attach the other end of the intraocular lens by opening the other pair of pincer arms and grasping iris tissue therebetween. Thus, this is a cumbersome process because the intraocular lens is constructed as a unitary member and the pairs of pincer arms are not independent from one another. Because both pairs of pincer arms are integrally and inseparably connected to the optical portion, the one pair of pincer arms can not be moved independently from either the other pair of pincer arms or from the optical portion itself. Thus, there is not a great deal of latitude in the insertion and anchoring of the intraocular lens in the eye due to the unitary construction of the intraocular lens.

Further, if the natural lens of the eye needs to be removed because of a condition, such as cataracts, etc., then an intraocular lens is implanted and is used in place of the natural lens. This is yet another use of an intraocular lens and it will be appreciated that the aforementioned uses/applications for the intraocular lens are merely exemplary and not limiting since there are other uses/applications.

What has heretofore not been available is an intraocular lens that includes a foldable optical lens part and is also attached to the iris tissue in such a manner that overcomes the deficiencies associated with conventional intraocular lens anchoring designs.

### SUMMARY

An intraocular lens is provided and according to one embodiment includes a deformable lens portion (e.g., an optic) that has a pair of support posts extending outwardly from the lens portion. The support posts are arranged circumferentially around the lens portion and according to one embodiment, the posts are axially aligned with one another with the axis of the posts extending through the center of the lens portion. The support posts are preferably integrally formed with the lens portion and have a length that is less than the diameter of the lens portion.

The intraocular lens further includes a pair of retaining or anchoring elements for securing the lens portion within an anterior chamber of the eye. Each retaining element is independent from the lens portion and can be formed of resilient material so that the retaining element is permitted to flex and bend. Each retaining element has a grasping feature that permits the retaining element to grasp a portion of the anterior surface of the iris tissue to thereby anchor the retaining element to the iris tissue within the anterior chamber. The retaining element also serves to position the lens portion within the anterior chamber and also provides the means for coupling the lens portion. More specifically, the retaining element is configured to receive a free end of the post in a manner in which the post is removable from the retaining element and also has some degree of movement relative to the retaining element. For example, the retaining element can include an opening or slot that receives the post and thereby restricts the degree of movement of the lens portion during use, while at the same time permitting axially movement of the lens portion due to the posts ability to axially move within the openings.

The type of fit between the post and the retaining element can vary depending upon the type of application and the precise configuration of the posts and the retaining elements. In one embodiment, the retaining element is formed of two finger sections that are biased toward one another with their biased free ends (i.e., pincer tips) being in intimate contact in a closed position. The free ends are opened apart from one another and the iris tissue is grasped between the free ends due to the natural inwardly biasing characteristics of the fingers. The post is received within an opening or slot that is defined between the fingers. Once the post is disposed between the fingers and the opening force is removed, the fingers flex inwardly toward their original closed position and this results in the iris tissue being held while the post has some degree of movement within the opening of the retaining element. Permitting movement of the post within the opening is desirable since it permits the lens portion to be readily inserted and removed from the retaining elements, thereby permitting easy updating or removal of the lens portion. In another embodiment, the post is not held by a tight frictional fit within the opening but rather is permitted to move freely within the opening while at the same time, a portion of the grasped iris tissue can extend into the opening and into contact with the post. Accordingly, the dimensions of the opening should be such that the opening can accommodate not only the post but also this grasped or pinched iris tissue.

The present intraocular lens overcomes many of the deficiencies associated with conventional intraocular lenses. For example, the incision that is needed to receive the present intraocular lens is smaller than incisions required to receive intraocular lenses that have a rigid lens portion and are formed as a unitary structure with the means for securing the lens portion to the iris tissue being formed as an integral part of the lens portion. Because the retaining elements in the present intraocular lens are separate and independent from the lens portion and the lens portion is formed of a deformable or foldable material, the dimensions of the incision are less. Furthermore, the independent nature of the retaining elements permits the retaining elements to be first positioned and secured to the iris tissue within the anterior chamber, of the eye prior to insertion of the lens portion. This reduces the complexity of the surgical procedure since the surgeon can fix the retaining elements without any interference from the optical portion.

Other features and advantages of the present invention will be apparent from the following detailed description when read in conjunction with the accompanying drawings.

### BRIEF DESCRIPTION OF THE DRAWING FIGURES

The foregoing and other features of the present invention will be more readily apparent from the following detailed description and drawings of illustrative embodiments of the invention in which:

FIG. 1 is a perspective view of an intraocular lens according to one embodiment implanted in an eye shown in partial section;

FIG. 2 is a side perspective view of the intraocular lens of FIG. 1;

FIG. 3 is a perspective view of a resilient ring being attached to iris tissue by grasping the tissue between resilient fingers thereof and a second resilient ring being inserted into an anterior chamber of the eye for attachment to the iris tissue;

FIG. 4 is a perspective view of a lens portion being inserted into the anterior chamber with one post of the lens portion being disposed through an opening in the resilient ring and an opposite post being spaced from the other resilient ring;

FIG. 5 is a perspective view of the lens portion of FIG. 4 being moved so that the opposite post is disposed through an opening in the other resilient ring, thereby coupling the lens portion to both resilient rings;

FIG. 6 is a cross-sectional view taken along the line 6-6 of FIG. 5

FIG. 7 is a cross-sectional side elevational view of the intraocular lens of FIG. 1 implanted in an eye;

FIG. 8 is a perspective view of the optical portion of FIG. 2 with a pair of retaining members according to another embodiment for anchoring the optical portion within the anterior chamber;

FIG. 9 is a cross-sectional view taken along the line 9-9 of FIG. 8;

FIG. 10 is a perspective view of an intraocular lens according to another embodiment; and

FIG. 11 is a perspective view, in partial cross-section, of the intraocular lens of FIG. 10 implanted in an eye.

### DETAILED DESCRIPTION OF PREFERRED EMBODIMENTS

Referring first to FIGS. 1-7 in which an intraocular lens 100 according to one embodiment is illustrated in an implanted position within an eye 10. The portions of the eye 10 that are illustrated include the cornea 12, iris 14, sclerotic tissue 16, vitreous 18, anterior chamber 20, pupillary area 21, chamber angle 22, and trabecular meshwork 24. A regular human lens is indicated at 26.

The intraocular lens 100 has an optical portion 110 that has a convex surface 112 that faces away from the lens 26 and a concave surface 114 that faces the lens 26. Thus, the illustrated optical portion 110 can be characterized as being a convex-concave lens portion. The intraocular lens 100 is intended to be surgically implanted into the eye 10 with the purpose of adding or subtracting the refractive power of the natural lens 26 in the eye 10 (phakic eye) for correcting a condition, such as myopia, hyperopia, presbyopia or astigmatism to provide an optical system with high predictability of the precalculated dioptic power. The intraocular lens can also be used to provide lost power in the aphakic eye. The optical portion 110 will typically have a shape that is complementary to the natural lens 26 and therefore, the optical portion 110 will likely have a circular or oval shape.

According to one aspect of the present application, the optical portion 110 is a deformable lens part that is formed of a suitable material that permits the optical portion 110 to be foldable. By constructing the optical portion 110 of such a material, the dimensions of the incision that is formed in the eye to receive the intraocular lens 100 can be reduced since the optical portion 110 can be folded, thereby reducing the overall dimensions of the intraocular lens 100.

There are a number of materials that can be used to form the deformable optical lens part 110 including, but not limited to, silicon elastomer, hydrogel polymer, and collagen containing polymer material, organic or synthetic gel compounds, polyurethane elastomer, or other suitable biocompatible materials. In one embodiment, the deformable optical lens part 110 is formed of a material that is disclosed in U.S. Pat. No. 6,387,127, which is hereby incorporated by reference in its entirety. More specifically, the deformable optical lens part 110 can be formed of a soft, especially swellable material, such as pHEMA (polyhydroxyethyl methacrylate) or a copolymer thereof, and in one particular embodiment, the deformable optical lens part 110 can be formed of a HEMA-MMA copolymer or a HEMA-ethyl methacrylate, or polymethylmethacrylate (PMMA). It will be understood that the aforementioned list is not exhaustive by any means and additional polymers, that are suitable for the intended application, can be used.

The intraocular lens 100 also has a pair of elongated arms or posts 120 that extend outwardly from the optical lens part 110. Accordingly, a first end 122 of the post 120 is attached to the optical lens part 120. A second end 124 of the post 120 represents a free distal end of the post 120. The posts 120 are preferably integrally attached to the optical lens part 110 at their first ends 122 and in one embodiment, the posts 120 and the optical lens part 110 can be formed at the same time during a fabrication process, such as a molding process, or the posts 120 and the optical lens part 110 can be constructed from a lens blank using conventional techniques or the posts 120 can be attached to lens part 110 after the lens part 110 is formed using conventional techniques, i.e., adhesives, fuse/melt bonding, etc. In the illustrated embodiment, each post 120 has a generally circular cross-section; however, the post 120 can have any number of other cross-section shapes. Each post 120 has a predetermined length to permit the intraocular lens to be easily inserted and implanted into the eye 10, as will be described in greater detail hereinafter.

Preferably, the posts 120 are axially aligned with one another and in one embodiment, the axis of the posts 120 passes through the center point of the optical lens part 110. However, it will be appreciated that the posts 120 do not have to be axially aligned since one post 120 can be axially offset from the other post 120 circumferentially about the lens part 110. The posts 120 can be formed of the same material that is used to form the optical lens part 110 or the posts 120 can be formed of a different material that is slightly more rigid than the material used to form the foldable optical lens part 110. The posts 120 can be formed of any number of biocompatible materials that are suitable for the intended purpose. For example, the posts 120 can be formed of polyurethane, polypropylene, polyimide, polymethylemethacrylate (PMMA), or other suitable biocompatible material, including the materials disclosed above with reference to those materials that are suitable for forming the optical lens part 110.

The intraocular lens 100 further includes a pair of retaining elements (e.g., resilient clamps) 130 that are designed to anchor the optical lens part 110 within the eye 10 so as to provide an effective implantation of the intraocular lens 100 within the eye 10 for the purpose of adding or subtracting the refractive power of the natural lens 26 or replace the natural lens 26 if it has been removed. The pair of retaining elements 130 are constructed to receive and anchor the posts 120 relative to the iris tissue (generally indicated at 14) and thus serve as a means for anchoring the optical lens part 110 within the eye 10. Preferably, the retaining elements 130 are disposed within iris tissue 14 that does not freely move during normal eye movement (i.e., normal contraction and dilation of the iris tissue 14). In other words, the outer portions of the iris tissue 14 represent sections of the iris tissue that do not move during normal eye movement. It is desirable to anchor the retaining elements 130 in this tissue since this type of anchoring enables the optical lens part 110 to remain in a set place above the natural lens 26 (when the lens 26 is in place) and therefore, undesirable movement of the lens part 110 does not occur even when the inner section of the iris tissue 14 undergoes normal movement.

According to one embodiment, each retaining element 130 is a resilient pinching member having first and second fingers 132, 134 that are spaced apart and define an opening 140 at their upper portions and are bent along intermediate sections so that the first and second fingers 132, 134 are spaced substantially parallel from one another along their lower portions. In a closed position, the first and second fingers 132, 134 are placed in intimate contact with one another at their lower portions and terminate in first and second pincer tips 136, 138, respectively. The retaining element 130 is a biased member such that in the closed position, the first and second fingers 132, 134 are biased towards one another into intimate contact. Preferably, when the first and second fingers 132, 134 intimately contact one another in the closed position, the opening 140 is a closed opening or a substantially closed opening and in one embodiment, the opening 140 is generally circular in shape; however, other shapes are possible, including oval, oblong, square, triangle, etc., so long as the shape is complementary to the shape of the post 120 that is received therethrough.

To secure the retaining ring 130 to the iris tissue 14, the fingers 132, 134 are opened by pulling apart the lower portions of the fingers 132, 134. As the fingers 132, 134 are opened, the dimensions of the opening 140 increase since the opening 140 is defined by the upper portions of the fingers 132, 134. It will also be appreciated that opening the fingers 132, 134 results in the opening 140 losing its closed nature since opening the fingers 132, 134 causes separation of the structure that defines the closed opening 140. After opening the fingers 132, 134, a portion of the iris tissue 14 is received between the fingers 132, 134 and more particularly, between the pincer tips 136, 138 thereof. The iris tissue 14 is effectively gripped and held between the pincer tips 136, 138 due to the biasing force which is applied to the iris tissue 14 by the pincer tips 136, 138. In this gripped position, the opening 140 is no longer a closed opening; however, the fingers 132, 134 are only spaced apart slightly and therefore the post 120 is not permitted to travel down between the spaced apart lower portions of the fingers 132, 134 (that are substantially disposed parallel to one another). In other words, the post 120 is confined within the opening 140 and according to a preferred embodiment, the dimensions of the opening 140 in the gripped position (where the iris tissue 14 is securely pinched between the pincer tips 136, 138) are slightly greater than the dimensions of the post 120. More specifically, the diameter of the post 120 is slightly less than the diameter of the opening 140 when the retaining element 130 is in the gripped position and therefore, the post 120 can move slightly within the opening 140. The posts 120 are thus permitted to axially move within the openings 140 while the radial movement thereof is restricted by the structure that forms the opening 140. This is one of the advantageous of the present intraocular lens 100 since it permits the optical part 110 to be easily coupled to the retaining elements 130 and also permits the optical part 110 to be easily and independently removed from the retaining elements 130, as will be described below. The retaining elements 130 should be spaced apart a predetermined distance so as to properly locate the optical part 110 within the anterior chamber 20.

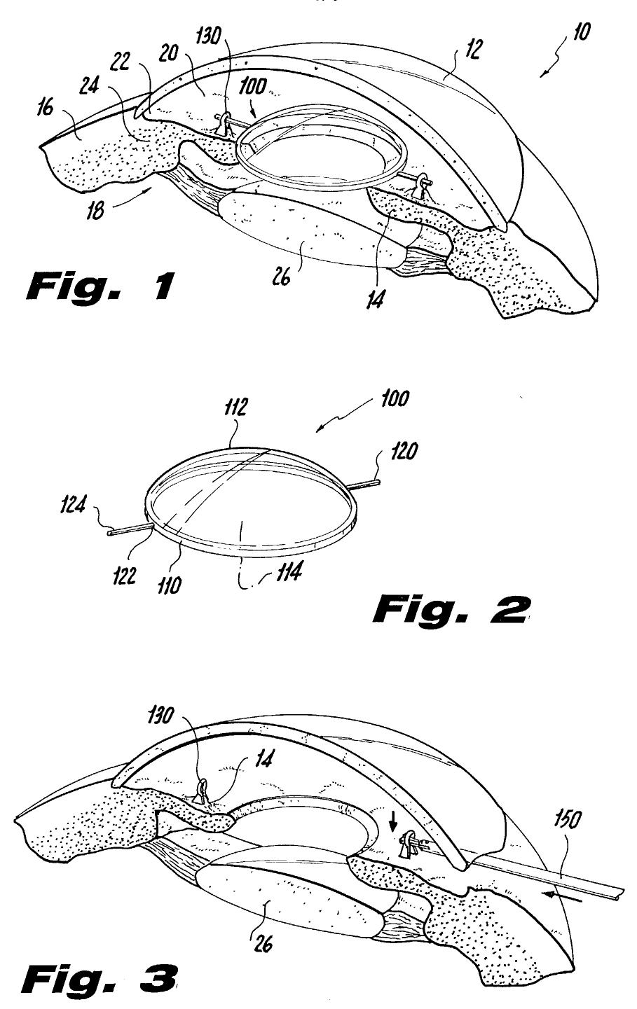

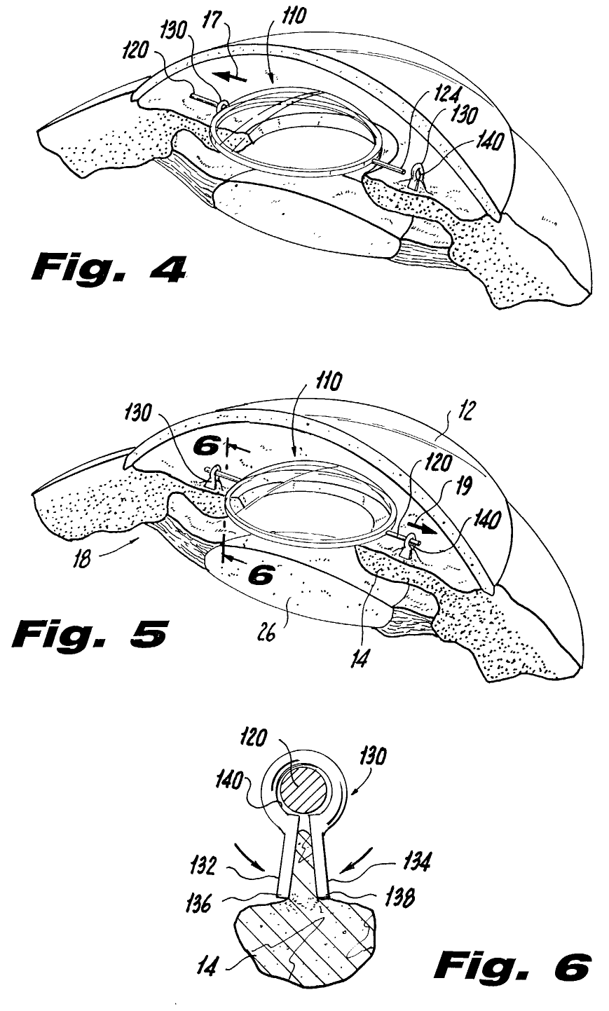

There are a number of ways to couple the optical portion 110 to the retaining elements 130. For example and as shown in FIGS. 3-7, the pair of retaining elements 130 can each be secured to the iris tissue 14 with the retaining elements 130 being spaced apart from one another and at predetermined locations relative to the pupillary area 21. In this attachment method, the retaining elements 130 are initially placed at locations where it is desired for the retaining elements 130 to remain after the optical portion 110 has been coupled to the retaining elements 130. As previously mentioned, it is preferred that the retaining elements 130 are anchored within iris tissue 14 that does not freely move during normal eye movement. Accordingly, the retaining elements 130 are preferably disposed in the outer sections of the iris tissue 14 as opposed to inner sections that move to effectuate contraction and dilation of the iris tissue 14.

In this embodiment, after each retaining element 130 has been secured to the iris tissue 14 by opening the first and second fingers 132, 134 and grasping the iris tissue 14 between the first and second pincer tips 136, 138, one post 120 is first inserted into the opening 140 of the respective retaining element 130 by receiving the second end 124 of the post 120 into the opening 140. Any number of conventional tools (such as tong-like tool 150 of FIG. 3 or the like) can be used to insert the retaining element 130 into the anterior chamber 20 and then open the fingers 132, 134 and then ultimately, grasp iris tissue 14 between the first and second pincer tips 136, 138.

Preferably, the length of the post 120 is such that the post 120 is slid through the opening 140, in the direction of arrow 17, until the retaining element 130 is in close proximity to the optical portion 110 as shown in FIG. 4. In this position, the lengths of the posts 120 have been configured so that the opposite post 120 has cleared the retaining element 130 and its second end 124 is disposed between the two retaining elements 130. Because the second end 124 has cleared the other retaining element 130, it can easily be inserted into the opening 140 of the retaining element 130 by simply moving the optical lens 110 back toward the opposite retaining element 130, in the direction of arrow 19 as shown in FIG. 5, until the second end 124 of the post 120 is received within the opening 140 of the respective retaining element 130. At the same time, the other post 120 remains disposed within the opening 140 of the other retaining element 130.

There are a number of devices that are available for inserting the optical lens part 110 through a small incision. For example, the flexible, foldable nature of the optical lens part 110 permits the optical lens 110 to be at least partially folded into an elongated injector device which carries the folded optical lens part 110 at a tip portion thereof and then discharges the optical lens part 110 once the tip portion is within the anterior chamber 20. This is merely one manner of placing the optical lens part 110 within the anterior chamber 20 so that it can be coupled to the retaining elements 130; however, it will be appreciated that there are a number of other different devices and techniques for accomplishing the same result.

Once both second ends 124 of the posts 120 are received within the openings 140, the optical portion 110 is effectively anchored to the iris tissue 14 and is positioned directly in front of the natural lens 26. The lengths of the posts 120 are such that the second end 124 of each post preferably extends a predetermined distance beyond the retaining element 130 when the optical portion 110 is fixed within the anterior chamber 20 by coupling the posts 120 to the retaining elements 130.

This method of attaching the intraocular lens 100 is much simpler than the methods used to attach the conventional larger, unitary intraocular lenses. More specifically, the intraocular lens disclosed in the '319 patent requires each end of the lens to be fixed by opening the pincer arms of each respective end after the lens has been inserted through the incision and then grasping the iris tissue at one end before the grasping the tissue at the other end. Because the optical lens part is integral with the pincer arms, the surgical technique of fixing one end by opening the pincer arms to grasp the iris tissue and then doing the same at the other end requires the surgeon to perform these steps with the optical lens part in place in between the pairs of pincer arms. In other words, the attachment of each end is made more difficult and is a more time consuming task due to the optical lens part being present. In contrast, the present intraocular lens 100 has independent attachment means (e.g., retainer elements 130) from the optical lens part 110 and therefore, the retaining elements 130 can be first positioned within the eye 10 without having to be concerned about the optical lens part 110. This greatly reduces the complexity of this surgical procedure and accordingly, reduces the time need to perform the procedure since the optical lens part 110 is not in the way when the retaining elements 130 are attached to the iris tissue 14.

Alternatively, the intraocular lens 100 can be implanted by having one of the retaining elements 130 already coupled to the post 120 by having the post 120 received within the opening 140. The retaining element 130 that is already coupled to the post 120 can then be fixed to the iris tissue 14 by using tool 150 to open the first and second fingers 132, 134 and then grasping the iris tissue 14 between the first and second pincer tips 136, 138 to securely anchor one end of the intraocular lens 100. The other retaining element 130 is then fixed to the iris tissue 14 by inserting the iris tissue 14 into the anterior chamber 20 and then fixing the retaining element 130 to the iris tissue 14 at its predetermined location by grasping the iris tissue 14 between the respective first and second pincer tips 136, 138. Once the other retaining element 130 is fixed to the iris tissue 14, the respective post 120 is then received within the opening 140, resulting in the coupling of the optical lens part 110 to the retaining elements 130 due to the posts 120 being receiving in the openings 140. The process can also be varied by first inserting and securing one retaining element 130 to the iris tissue 14 and then inserting the optical lens part 110 with the other retaining element 130 being disposed along the post 120. The other post 120 is then inserted into the opening 140 of the retaining element 130 that was first fixed to the iris tissue 14 and then the second retaining element 130 is fixed to the iris tissue 14, thereby fixing the optical lens part 110 within the anterior chamber 20.

If the surgeon wishes to move the intraocular lens 100 by repositioning one or more retaining elements 130 after it has been coupled to the retaining elements 130, the surgeon can simply adjust the initial positions of the retaining elements 130 by opening the first and second pincer fingers 132, 134 of one or more of the retaining elements 130 and then making the necessary adjustment and regrasping the iris tissue 14. It will be appreciated that because of the coupling action between the post 120 and the retaining element 130, the retaining element 130 can be repositioned without necessarily having to remove the respective post 120 from the retaining element 130 since the post 120 can slide within the opening 140. Of course, the surgeon can reposition the intraocular lens 100 by removing one or more posts 120 from their respective openings 140 and then opening the first and second fingers 132, 134 to permit repositioning thereof. After the retaining element 130 is properly repositioned and is in a grasping relationship with the iris tissue 14, the second end 124 of the post 120 is reinserted into the opening 140.

As previously mentioned, the dimensions of the posts 120 relative to the dimensions of the openings 140 should be such that some movement of the posts 120 within the openings 140 is permitted to accommodate tissue movement, while excessive movement is prevented since this type of movement can cause undesirable movement of the optical lens part 110. Permitting some movement of the posts 120 within the retaining elements 130 provides an advantage over the conventional designs (such as the design in the '319 patent) due to the posts 120 ability to tolerate the natural movements of the iris tissue 14. In effect, the sliding (i.e., axial movement) of the posts 120 within the openings 140 is a self-adjusting mechanism to accommodate natural expansion and contraction of the iris tissue 14. In any event, the components of the intraocular lens 100 can easily be disassembled by slidably removing the post 120 from the opening 140 and then opening the first and second fingers 132, 134 to disengage the retaining element 130 from the iris tissue 14. The ease of removing the optical lens part 110 permits the optical lens part 110 to be changed if conditions warrant such change (e.g., prescription needs to be updated or lens part 110 is damaged, etc.). In direct contrast, conventional intraocular lenses are typically designed as a one piece assembly in which the retaining means are formed as part of the lens assembly and therefore, removal of the optical lens requires removal of the entire intraocular lens. This requires the retaining means to be disengaged from the iris tissue so as to free the intraocular lens and permit its removal. Unfortunately, this can be a difficult and time consuming task since over time the retaining means may become more difficult to remove from the iris tissue. Also, the insertion of the new intraocular lens into the anterior chamber 20 requires that the new intraocular lens be anchored to the iris tissue.

According to one exemplary embodiment, the optical lens part 110 has a diameter of about 6 mm and each post 120 has a length of about 1.0 mm to about 2.5 mm and a cross-sectional diameter of about ½ mm to about 1.5 mm. The retaining element 130 has an outside diameter of about 1.0 mm to about 2.0 mm and an inside diameter between about 3/4 mm to 1.5 mm. The dimensions of the opening 140 are thus defined by the inside diameter of the element 130 with the cross-section of the post 120 being selected so that the post 120 can be received within the opening 140 with a slight gap formed therebetween. In one embodiment, the post 120 has a cross-section that is about ½ mm less than the diameter of the opening 140, thereby creating about ¼ mm clearance between the post surface and the retaining element 130. The thickness of the retaining element 130 should be optimized to be as less invasive as possible; however, the thickness should be sufficient so that the first and second fingers 132, 134 are naturally biased closed and an applied force is needed to separate the fingers 132, 134 and removal of this force results in the fingers 132, 134 being biased back into intimate contact with one another. It will be clearly understood that the aforementioned dimensions are merely exemplary in nature and that the intraocular lens 100 can have dimensions outside of the aforementioned dimensions depending upon a number of different variables, including the precise construction of the retaining elements 130, the dimensions of the opening 140, etc. Furthermore, opening 140 is not limited to having a circular shape since it can be any number of other shapes, such as oval, oblong, square, triangular, etc., so long as the shape of the posts 120 are complementary and a large gap is not formed between the posts 120 and the retaining elements 130 when the posts 120 are received in the openings 140 when the retaining elements 130 are in the gripped position.

In the exemplary embodiment where the optical lens part 110 has a diameter of about 6 mm, the distance between the retaining elements 130 is about 8 mm. It will be appreciated that the retaining elements 130 are preferably spaced apart a sufficient distance to (1) properly locate and center the optical part 110 within the anterior chamber 20; and (2) be located in outer regions of the iris tissue so that the retaining elements 130 are not anchored in iris tissue that constantly moves during normal eye movement.

One of the advantages of the present intraocular lens 100 is that the size of the incision that is formed by the surgeon to permit implantation of the intraocular lens 100 is reduced relative to traditional lens constructions. The incision dimensions can be reduced because the optical lens part 110 is preferably formed of a material that allows the optical lens part 110 to be deformable (foldable) and also the intraocular lens 100 does not include a large body having integral attachments means as a part thereof; but rather, the intraocular lens 100 has small posts 120 and retaining elements 130 that are independent from the optical lens part 110. According to one embodiment, the deformable optical lens part 110 is configured to be inserted through a small incision in the eye 10, preferably under 3 mm, more preferably under 2.5 mm and most preferably under 2 mm. The action of the first and second pincer tips 136, 138 permits the post operative decentralization and dislocation rate of the intraocular lens 100 to approach zero. In other words, the retaining elements 130 provide an excellent means for fixedly retaining the intraocular lens 100 within the anterior chamber 20.

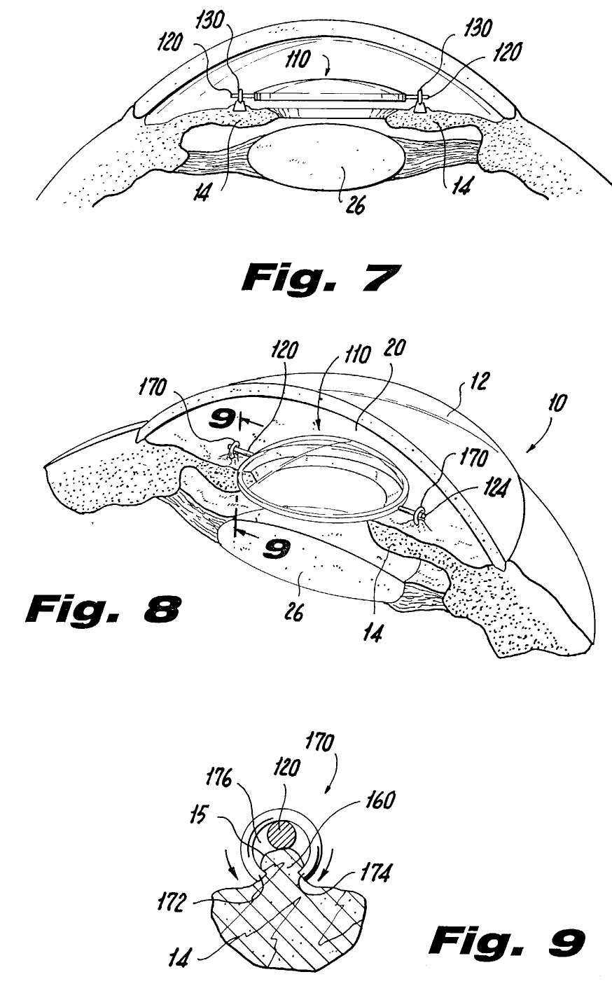

Referring now to FIGS. 8 and 9 in which yet another embodiment is illustrated. In this embodiment, the intraocular lens includes the optical portion 110 of FIGS. 1-7 but includes resilient retaining rings 170 as the means for anchoring the optical portion 110 within the anterior chamber 20 to provide an effective implantation of the intraocular lens 100 within the eye 10. As with the retaining elements 130, the retaining rings 170 are constructed to receive and anchor the optical lens part 110 within the eye 10. The retaining ring 170 is a resilient pinching member terminating in a first pincer tip 172 at a first end and a second pincer tip 174 at a second end. The retaining ring 170 also has an opening or slot 176 formed therethrough for receiving the free second end 124 of one post 120. According to one embodiment, the opening 176 is formed to have dimensions that are greater than the dimensions of the second end 124 of the post 120 so that the second end 124 can be received within the opening 176 in a movable manner so that the post 120 can slide into or out of the opening 176 when an appropriate force is applied. Preferably, the opening 176 is sized so that it can accommodate not only the post 120 but also some tissue that will likely be received between the first and second pincer tips 172, 174. In the illustrated embodiment, the retaining ring 130 is a generally circular, curved structure with split ends in the form of the first and second pincer tips 172, 174.

In a closed position, the first and second pincer tips 172, 174 are spaced apart from one another in close relationship or the first and second pincer tips 172, 174 are in intimate contact with one another (i.e., an abutting relationship) in the closed position. The resilient, flexible nature of the retaining ring 170 permits the first and second pincer tips 172, 174 to be opened relative to one another by pulling the first and second pincer tips 172, 174 apart from one another or by inserting an object (e.g., the tong-like tool 150 in FIG. 3) between the first and second pincer tips 172, 174 and then applying an outward force against one or both of the first and second pincer tips 172, 174 to form a gap 160 therebetween.

To fix the intraocular lens within the anterior chamber 20, the first and second pincer tips 172, 174 of each of the retaining rings 170 are opened and manipulated so that they grasp the iris tissue 14. When the iris tissue 14 is grasped by the first and second pincer tips 172, 174, the grasped iris tissue 14 forms a bulge 15 that is disposed between the spaced apart first and second pincer tips 172, 174 and protrudes at least slightly into the opening 176. The resilient nature of the retaining ring 170 causes the first and second pincer tips 172, 174 to return to the closed position once the opening force is removed and therefore, after the iris tissue 14 is disposed in the gap 160 and the first and second pincer tips 172, 174 are released (i.e., the force is removed), the iris tissue 14 will be effectively grasped between the first and second pincer tips 172, 174. The first and second pincer tips 172, 174 are covered by the iris tissue 14 when the retaining ring 170 engages the iris tissue 14. Because the retaining ring 170 is preferably formed of a resilient material, it can flex and fold over if a force is applied in that direction. In other words, while the retaining ring 170 may stand upwardly from the iris tissue 14 after the first and second pincer arms 172, 174 (located at a bottom portion thereof) grasp the iris tissue 14, the retaining ring 170 is flexible in a number of directions. Of course, the retaining ring 170 can be made more rigid based upon material selection.

Any number of different methods can be selected to provide an effective coupling between the posts 120 and the retaining rings 170. For example, the relative dimensions of the openings 176 and the posts 120 can be selected so as to provide a close intimate fit between the posts 120 and the retaining rings 170. Because a portion of the grasped iris tissue 14 likely extends into the opening 176 as shown in FIG. 9, the dimensions of the second end 124 of the post 120 should be undersized relative to the dimensions of the opening 176 to permit the second end 124 to be slidably coupled to the retaining element 176, while at the same time, the iris tissue 14 is accommodated within the opening 176.

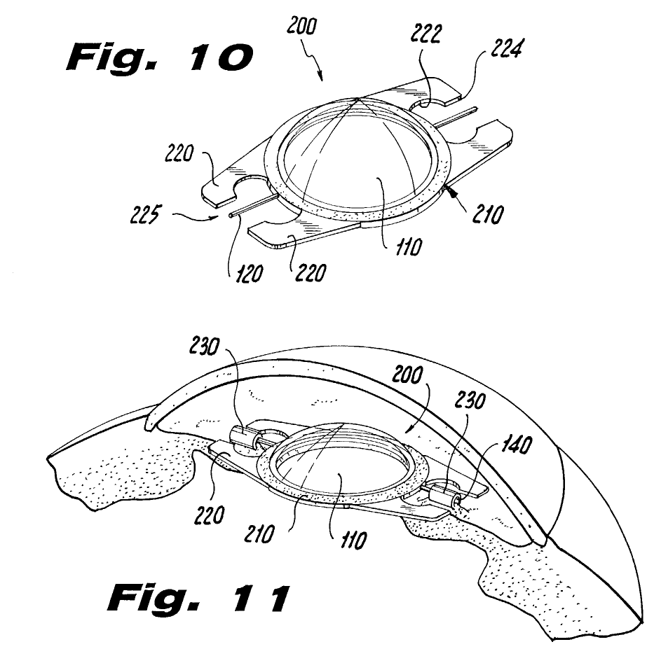

Referring now to FIGS. 10-11 in which an intraocular lens 200 is illustrated according to another exemplary embodiment. The intraocular lens 200 is very similar to the intraocular lens 100 with the exception that the intraocular lens 200 has a peripheral base 210 that is formed around the optical lens part 110. The peripheral base 210 can be formed of the same material as either the optical lens part 110, the posts 120 or retaining elements or the peripheral base 210 can be formed of a different material compared to one or more of the aforementioned components. For example, the peripheral base 210 can be formed of a non-transparent material so as to prevent glare, as illustrated in FIGS. 10-11. The intraocular lens 200 also includes a pair of extensions or arms (wings) 220 that are formed at each end thereof. Each arm 220 is attached at one end to the peripheral base 210 and has a curved section 222 that terminates in a distal end 224. The arms 220 are spaced apart from one another with one post 120 extending between the pair of arms 220. In one exemplary embodiment, the arms 220 are curved such that the distal ends 224 face another with a gap 225 formed therebetween. The arms 220 are preferably constructed so that during normal application they seat against iris tissue 14 and therefore act as a stabilizing structure that prevents undesirable movement of the optical portion 110 relative to the iris tissue 14.

The precise relationship between the posts 120 and the arms 220 is variable due to design choices, relative dimensions, etc. The length of the post 120 can be such that the post 120 does not extend beyond the spaced apart pair of arms 220 or the intraocular lens 200 can be constructed so that the second end 124 of the post 120 does extend beyond the arms 220. Preferably, the arms 220 are formed substantially in the same plane as the posts 120; however, the arms 220 can be disposed in a plane that is distinct from the plane containing the posts 120 in order for the arms 220 to seat against the iris tissue 14 during normal use to provide extra stability (i.e., lateral stability) and prevent the optical portion 110 from freely rotating about an axis containing the posts 120.

Retaining elements 230 are similar to retaining elements 170 with the exception that the length of the retaining elements 230 has been increased so that the body of the retaining elements 230 is more substantial and the portion defining the opening 140 is tube-like in nature. In this embodiment, the length of the retaining element 230 is from about 1.5 mm to about 2.5 mm. By increasing the body dimensions of the retaining elements 230, additional biasing force can be provided and also the robustness of the grasping/pinching action is increased to ensure that the grasped iris tissue 14 does not become free. It will be understood that the retaining elements 130 shown in FIGS. 1-7 can also be formed to have an increased length such as the retaining elements 230 in an effort to increase the robustness and gripping power of the retaining elements. When constructed in this manner, the opening 140 is defined in a generally tube-like structure that has elongated finger 132, 134 extending therefrom.

Any of the exemplary intraocular lenses disclosed herein can be fabricated so that they act as a conventional intraocular lens, a toric intraocular lens, or a multifocal intraocular lens. In other words, the optical lens part 110 can be constructed to have any number of characteristics depending upon the patient's needs. For example, the optical lens part 110 can have several regions with different powers. Moreover, the outer surface of the optical lens part 110 can curved (e.g., convex as in FIG. 1) or it can be flat. Because the inner surface of the optical portion is concave, the optical portion is positioned a safe distance away from the natural lens 26 and also the dimensions (i.e., standing height) of the retaining members and the location where the retaining members grasp the iris tissue 14 are selected so that that retaining members are spaced a sufficient distance from the cornea 12 so that it is unlikely that the retaining members or any other portion of the intraocular lens will make contact therewith during normal use of the intraocular lens.

While the invention has been particularly shown and described with reference to preferred embodiments thereof, it will be understood by those skilled in the art that various changes in form and details can be made therein without departing from the spirit and scope of the invention.

What is claimed is:

1. An intraocular lens, comprising:
a deformable optic;
first and second support posts spaced circumferentially about the optic and extending outwardly therefrom; and
a pair of retaining elements for securing the optic within an anterior chamber of the eye by grasping a portion of an anterior surface of iris tissue, each retaining element having flexible first and second pincer tips that are normally in an abutting or a close abutting relationship, wherein each retaining element is secured to the anterior surface by opening the first and second pincer tips and pinching the anterior surface of the iris tissue, each of the retaining elements defining an opening for receiving one of the first and second support posts to couple the optic to the pair of retaining elements.

2. The intraocular lens of claim 1, wherein the optic is generally circular in shape.

3. The intraocular lens of claim 1, wherein each of the first and second support posts has a length that is less than the diameter of the optic.

4. The intraocular lens of claim 1, wherein the first and second support posts are axially aligned with one another.

5. The intraocular lens of claim 4, wherein the axis containing the first and second support posts extends through a center of the optic.

6. The intraocular lens of claim 1, wherein each retaining element has an arcuate shape with the first and second pincer tips being split ends of the retaining element.

7. The intraocular lens of claim 1, wherein the retaining element is formed of a resilient material so that it is naturally biased to a closed position in which the first and second pincer tips are in an abutting relationship or close to an abutting relationship, wherein the first and second pincer tips are opened by applying a force to create a gap therebetween which receives the anterior surface of the iris tissue and upon removal of the force, the first and second pincer tips move toward the closed position, resulting in the portion of the anterior surface being pinched.

8. The intraocular lens of claim 1, wherein each of the retaining elements comprises a bent member formed of two opposing fingers that are joined at their upper portions, the upper portions defining a shaped slot for receiving one of the posts, the fingers having lower portions that are biased together in a closed position and are openable to permit reception of the portion of the anterior surface.

9. The intraocular lens of claim 8, wherein in an open position, the distance between the fingers is less than the diameter of the post received within the opening so that the post is prevented from moving from the shaped slot into a position between the lower portions of the fingers.

10. The intraocular lens of claim 8, wherein a diameter of the shaped slot is between about 10% to about 20% greater than a diameter of the post.

11. The intraocular lens of claim 1, further including:
a frame formed around a periphery of the deformable optic, the first and second posts being attached to the frame and extending outwardly therefrom, wherein the frame has at least a peripheral edge section surrounding a peripheral edge of the optic that is formed of a material that has different optical properties compared to the optic.

12. The intraocular lens of claim 11, wherein at least the peripheral edge section is formed of a non-transparent material so as to prevent glare.

13. The intraocular lens of claim 11, wherein at least a portion of the peripheral edge section that has different optical properties is ring-shaped.

14. The intraocular lens of claim 11, wherein the frame includes a pair of arms formed at each end of the frame, each of the arms having a curved section that terminates in a distal end, the distal ends of one pair facing one another in a spaced apart manner

15. The intraocular lens of claim 14, wherein one of the posts extends between one pair of arms with a space being formed between the posts for receiving the retaining element.

16. The intraocular lens of claim 14, wherein the one post is disposed substantially in the same plane that contains the surrounding pair of arms.

17. An intraocular lens, comprising:
a deformable optic;
first and second support posts extending outwardly from the optic; and
a pair of resilient clamps that are independent from the optic for securing the optic within an anterior chamber of an eye by grasping a portion of an anterior surface of iris tissue between two resiliently biased fingers which have first and second pincer tips which are in an abutting or a close abutting relationship when the clamp is in a closed position, each of the resilient clamps having an opening defined therethrough for receiving one of the first and second support posts to effectively couple the optic to the resilient members, wherein one resilient clamp grasps iris tissue on one side of a pupillary area and the other resilient clamp grasps iris tissue on the other side of the pupillary area with the deformable optic extending across the pupillary area.

18. An intraocular lens, comprising:
a deformable optic;
first and second support posts spaced circumferentially about the optic; and
first and second pincer elements that are independent from the deformable optic, each of the first and second pincer elements including first and second pincer tips that are biased into a closed position where the first and second pincer tips face one another, the first and second pincer elements being formed of a resilient material to permit opening of the first and second pincer tips so as to permit an anterior surface of the iris tissue to be received between and grasped by the first and second pincer tips, each of the first and second pincer elements receiving one of the first and second support posts in a manner in which the respective support post is removable from the pincer element and can be slidably adjusted relative to the pincer element.

19. The intraocular lens of claim 18, wherein the optic is generally circular in shape.

20. The intraocular lens of claim 18, wherein each of the first and second posts has an annular cross-section.

21. The intraocular lens of claim 18, wherein the first and second support posts are axially aligned with one another.

22. The intraocular lens of claim 18, wherein each of the first and second pincer elements comprises a split end resilient ring with one of the first and second support posts being received within an opening defined the ring, the grasped anterior surface of the iris tissue extending into the opening.

23. The intraocular lens of claim 18, wherein each of the pincer elements is a bent clip member having two opposing finger sections that terminate in the first and second pincer tips, the first and second pincer tips being biased into intimate contact with one another in the closed position, wherein one of the first and second support posts is received within an opening formed between the first and second fingers.

24. The intraocular lens of claim 18, wherein the deformable optic is a multi-focal lens.
